(19) United States
(12) Patent Application Publication
(10) Pub. No.: US 2024/0241118 A1
(43) Pub. Date: Jul. 18, 2024

(54) AUTOANTIBODIES FOR USE IN IDENTIFYING DISEASE

(71) Applicant: The Johns Hopkins University, Baltimore, MD (US)

(72) Inventors: Antony Rosen, Pikesville, MD (US); Brittany Adler, Baltimore, MD (US); Livia A. Casciola-Rosen, Pikesville, MD (US)

(21) Appl. No.: 18/289,332

(22) PCT Filed: Jan. 24, 2022
(86) PCT No.: PCT/US2022/013468

§ 371 Date: Nov. 2, 2023

Related U.S. Application Data
(60) Provisional application No. 63/188,758, filed on May 14, 2021.

(51) Int. Cl. G01N 33/564; C12Q 1/6841; C12Q 1/6851; C12Q 1/6883

(52) U.S. Cl. CPC G01N 33/564; C12Q 1/6883; C12Q 1/6841; C12Q 1/6851; G01N 2800/12; G01N 2800/24

(57) ABSTRACT

Provided herein are methods of identifying a disease in a subject that include: (a) providing a biological sample from a subject; and (b) detecting an autoantibody that targets an antigen present in a telomerase/shelterin complex in the biological sample, thereby identifying a disease in the subject.

### CROSS-REFERENCE TO RELATED APPLICATIONS

This application claims priority to U.S. Provisional Patent Application No. 63/188,758, filed on May 14, 2021. The disclosure of this prior application is considered part of the disclosure of this application, and is incorporated in its entirety into this application.

### FEDERALLY SPONSORED RESEARCH OR DEVELOPMENT

This invention was made with government support under T32 AR048522, P30-AR053503, and P30-AR070254 awarded by the National Institutes of Health. The government has certain rights in the invention.

### TECHNICAL FIELD

The present disclosure relates to the field of biotechnology, and more specifically, to autoantibodies.

### BACKGROUND

Systemic sclerosis (SSc) is an autoimmune chronic fibrosing disease of unknown etiology that results in vasculopathy and multi-organ fibrosis. The disease is heterogeneous with a wide range of possible clinical manifestations that include skin thickening, interstitial lung disease (ILD), and Raynaud's phenomenon. The majority of SSc patients develop ILD, which has some clinical similarities with the progressive lung scarring seen in idiopathic pulmonary fibrosis (IPF). Telomere dysregulation has been observed in both SSc and IPF, although it remains unclear if there are common mechanistic pathways underlying telomere dysfunction in these diseases.

Autoantibodies targeting telomere-associated proteins in a subset of SSc patients are associated with short lymphocyte telomere length and lung disease. The specificity of these autoantibodies for SSc and IPF suggests that telomere dysfunction may have a distinct role in the pathogenesis of SSc and pulmonary fibrosis.

### SUMMARY

The present disclosure is based in part on the discovery that detection of an autoantibody that targets an antigen of a telomerase/shelterin complex in a subject can be used as a biomarker for identifying systemic sclerosis, interstitial lung disease, and fibrotic lung disease in the subject. Without wishing to be bound by any theory, it has been discovered that detection of an autoantibody targeting the telomerase/shelterin complex can be used as a biomarker to identify a disease in a subject.

Provided herein are methods of identifying a disease in a subject, the method including: (a) providing a biological sample from a subject; and (b) detecting an autoantibody that targets an antigen present in a telomerase/shelterin complex in the biological sample, thereby identifying a disease in the subject.

Also provided herein are methods of treating a disease in a subject, the method including: (a) providing a biological sample from a subject; (b)detecting an autoantibody that targets an antigen present in a telomerase/shelterin complex in the biological sample, thereby identifying a disease in the subject; and (c) administering a treatment to the subject, wherein the treatment is predicted to be beneficial to the subject based when the presence of the autoantibody is detected.

In some embodiments, the disease is systemic sclerosis (SSc), idiopathic pulmonary fibrosis (IPF), or interstitial lung disease (ILD). In some embodiments, the disease is a rheumatic disease. In some embodiments, the treatment comprises administration of a therapy selected from an anti-inflammatory medication, an immunosuppressive therapy, a drug therapy of vascular disease, an anti-fibrotic agent, an oxygen therapy, pulmonary rehabilitation, or a drug therapy for pulmonary fibrosis, and combinations thereof.

In some embodiments, the subject is a human. In some embodiments, the biological sample is blood or a blood fraction obtained from the subject. In some embodiments, the biological sample is sera obtained from the subject.

In some embodiments, the autoantibody is a monoclonal antibody. In some embodiments, the antigen comprises one or more of hTERT, TERF1, TERF2, POT1, TIN2L, and RAP1. In some embodiments, the method comprises detecting two or more autoantibodies. In some embodiments, the detecting step (b) comprises detecting the presence or absence of an interaction between the autoantibody and the antigen. In some embodiments, the detecting step (b) comprises an immunoprecipitation assay, an enzyme-linked immunosorbent assay (ELISA), or an immunoblotting assay.

In some embodiments, the method further comprises analyzing a second biological sample from the subject to measure telomere length in a cell from the second biological sample. In some embodiments, the biological sample and the second biological sample are the same. In some embodiments, the telomere length is measured by performing qPCR, Flow-FISH, or both. In some embodiments, the cell is a leucocyte. In some embodiments, the cell is a peripheral blood mononuclear cell (PBMC).

In some embodiments, the subject is identified to have the disease when the autoantibody is detected in the biological sample from the subject. In some embodiments, the subject is identified to not have the disease when the autoantibody is not detected in the biological sample from the subject.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIGS. 1A-1C show autoantibodies targeting the telomerase/shelterin complex in scleroderma.

FIGS. 1A-1B show results from immunoprecipitations (IPs) that were performed with patient sera (JH scleroderma cohort, n=200) using lysate made from HEK293 cells overexpressing hTERT-FLAG as input. IPs were detected by blotting with an anti-FLAG antibody. hTERT autoantibodies were found in 6/200 of the scleroderma patients (Scl 1-6) and 0/30 healthy controls. FIG. 1C shows IPs that were performed with patient sera using the six 35S-methionine-labeled shelterin proteins generated by in vitro transcription and translation. IPs with anti-FLAG was used for positive controls. At least one shelterin autoantibody was detected in 6/200 scleroderma patient sera. Results show, in total, 7/200 (3.5%) scleroderma patients either immunoprecipitated hTERT or had an autoantibody targeting at least one shelterin protein.

FIG. 2 is an exemplary image of IVTT IP's of 6 healthy control serum using the six shelterin IVTT products combined as input (POT1, TPP1, TIN2L, TERF1, TERF2, and RAP1). IP with anti-FLAG was used as positive controls. A total of 30 healthy control sera were screened for all six shelterin autoantibodies and none were positive.

FIG. 3 shows exemplary images of immunoblots of recombinant TERF1 protein to confirm the presence of TERF1 autoantibodies among patients positive by ELISA but negative by TERF1 IVTT IP. Representative images of immunoblots performed using patient sera (n=12 scleroderma (SSc) and n=12 controls) to blot recombinant his-tagged TERF1 protein are shown. Upper panels show data obtained using patient or control sera for blotting and lower panels show the blots reprobed with an anti-His monoclonal antibody.

FIG. 4 is a graph showing TERF1 autoantibodies detected by ELISA in healthy controls (n=78), the combined JH and UCSF scleroderma cohorts (Scl, n=442), rheumatoid arthritis (n=60), and myositis (n=60). Fisher's exact test was used to compare the frequency of TERF1 autoantibodies between different cohorts. *p<0.05

FIGS. 5A-5B show peripheral blood leukocyte telomere length measured by qPCR in 242 scleroderma patients from the University of California, San Francisco (UCSF) Scleroderma Center. FIG. 5A shows the relationship between leukocyte telomere length and age for TERF1 autoantibody positive (n=18) and negative (n=224) patients. FIG. 5B shows patients with TERF1 autoantibodies have a significantly shorter telomere length relative to the expected age-adjusted telomere length compared to patients without TERF1 autoantibodies. Statistics were performed using Wilcoxon rank-sum test, *p<0.05.

FIGS. 6A-6D are a set of graphs showing telomere length measured by Flow-FISH (flow cytometry and fluorescent in-situ hybridization) in lymphocytes and granulocytes of 6 TERF1 autoantibody-positive scleroderma patients and 10 TERF1 autoantibody-negative scleroderma patients. FIGS. 6A-6B show nomograms of telomere length relative to age in lymphocytes and granulocytes relative to a healthy control population depicted by percentiles. Patients with TERF1 autoantibodies are depicted in blue, and those without TERF1 autoantibodies are in red.

FIGS. 6C-6D show patients with TERF1 autoantibodies have shorter telomere lengths in lymphocytes (FIG. 6C) but not granulocytes (FIG. 6D) compared to TERF1 autoantibody-negative patients. Delta TL is the difference between the patient telomere length and the median telomere length of a healthy control population.

FIG. 7 is a Venn diagram showing overlap among TERF1 (n=22), U1RNP (n=17), and Ku (n=11) autoantibodies in the Hopkins (JH) scleroderma cohort. 40/200 (20%) of patients in the JH cohort had at least one of these autoantibodies.

FIG. 8 shows TERF1 autoantibodies detected by ELISA in healthy controls (n=78) and the University of California, San Francisco (UCSF) Idiopathic Pulmonary Fibrosis (IPF) cohort (n=152).

FIG. 9 shows the presence of TERF1 autoantibodies measured by ELISA in lupus (n=60) compared to healthy controls (n=78).

### DETAILED DESCRIPTION

The present disclosure is based in part on the discovery that detection of an autoantibody that targets an antigen of a telomerase/shelterin complex in a subject can be used as a biomarker for identifying systemic sclerosis, interstitial lung disease, and fibrotic lung disease in the subject. In some embodiments, the presence of an antibody targeting components of the telomerase/shelterin complex (e.g., hTERT, TERF1, TERF2, POT1, TPP1, TIN2L, and RAP1) can be used as a biomarker for systemic sclerosis and specifically for systemic sclerosis lung disease. In some embodiments, the presence of autoantibodies targeting telomere-associated proteins in systemic sclerosis and their association with short telomeres can provide insights into telomere dysfunction in systemic sclerosis. In some embodiments, the association of telomere-associated autoantibodies with interstitial lung disease in systemic sclerosis and the presence of these autoantibodies in idiopathic pulmonary fibrosis can support a role of telomere dysregulation in pulmonary fibrosis.

In some embodiments, provided herein are methods of identifying a disease in a subject, the method including: (a) providing a biological sample from a subject; and (b) detecting an autoantibody that targets an antigen of a telomerase/shelterin complex in the biological sample, thereby identifying a disease in the subject. In some embodiments, the disease is systemic sclerosis (SSc), idiopathic pulmonary fibrosis (IPF), or interstitial lung disease (ILD).

Various non-limiting aspects of these methods are described herein, and can be used in any combination without limitation. Additional aspects of various components of the methods described herein are known in the art.

As used herein, the term “antibody” refers to an agent that specifically binds to a particular antigen. In some embodiments, the term encompasses any polypeptide or polypeptide complex that includes immunoglobulin structural elements sufficient to confer specific binding. Exemplary antibodies include, but are not limited to monoclonal antibodies, polyclonal antibodies, and fragments thereof.

As used herein, the term “antigen” refers to an agent that binds to an antibody. In some embodiments, an antigen binds to an antibody and may or may not induce a particular physiological response in an organism. In general, an antigen may be or include any chemical entity such as, for example, a small molecule, a nucleic acid, a polypeptide, a carbohydrate, a lipid, a polymer (including biologic polymers [e.g., nucleic acid and/or amino acid polymers] and polymers other than biologic polymers [e.g., other than a nucleic acid or amino acid polymer]) etc. In some embodiments, an antigen is or comprises a polypeptide. In some embodiments, an antigen is or comprises a glycan. Those of ordinary skill in the art will appreciate that, in general, an antigen may be provided in isolated or pure form, or alternatively may be provided in crude form (e.g., together with other materials, for example in an extract such as a cellular extract or other relatively crude preparation of an antigen-containing source). In some certain embodiments, an antigen is present in a cellular context (e.g., an antigen is expressed on the surface of a cell or expressed in a cell). In some embodiments, an antigen is a recombinant antigen.

As used herein, the term “biological sample” refers to a sample obtained from a subject for analysis using any of a variety of techniques including, but not limited to, biopsy, surgery, and laser capture microscopy (LCM), and generally includes cells and/or other biological material from the subject. A biological sample can be obtained from a eukaryote, such as a patient derived organoid (PDO) or patient derived xenograft (PDX). The biological sample can include organoids, a miniaturized and simplified version of an organ produced in vitro in three dimensions that shows realistic micro-anatomy. Subjects from which biological samples can be obtained can be healthy or asymptomatic individuals, individuals that have or are suspected of having a disease (e.g., cancer) or a pre-disposition to a disease, and/or individuals that are in need of therapy or suspected of needing therapy.

Biological samples can include one or more diseased cells. A diseased cell can have altered metabolic properties, gene expression, protein expression, and/or morphologic features. Examples of diseases include inflammatory disorders, metabolic disorders, nervous system disorders, and cancer. Cancer cells can be derived from solid tumors, hematological malignancies, cell lines, or obtained as circulating tumor cells.

Biological samples can also include immune cells. Sequence analysis of the immune repertoire of such cells, including genomic, proteomic, and cell surface features, can provide a wealth of information to facilitate an understanding the status and function of the immune system. Examples of immune cells in a biological sample include, but are not limited to, B cells, T cells (e.g., cytotoxic T cells, natural killer T cells, regulatory T cells, and T helper cells), natural killer cells, cytokine induced killer (CIK) cells, myeloid cells, such as granulocytes (basophil granulocytes, eosinophil granulocytes, neutrophil granulocytes/hypersegmented neutrophils), monocytes/macrophages, mast cells, thrombocytes/megakaryocytes, and dendritic cells.

The biological sample can include any number of macromolecules, for example, cellular macromolecules and organelles (e.g., mitochondria and nuclei). The biological sample can be a nucleic acid sample and/or protein sample. The biological sample can be a carbohydrate sample or a lipid sample. The biological sample can be obtained as a tissue sample, such as a tissue section, biopsy, a core biopsy, needle aspirate, or fine needle aspirate. The sample can be a fluid sample, such as a blood sample, urine sample, or saliva sample. The sample can be a skin sample, a colon sample, a cheek swab, a histology sample, a histopathology sample, a plasma or serum sample, a tumor sample, living cells, cultured cells, a clinical sample such as, for example, whole blood or blood-derived products, blood cells, or cultured tissues or cells, including cell suspensions.

As used herein, the term “subject” refers to an organism, typically a mammal (e.g., a human). In some embodiments, a subject is suffering from a relevant disease, disorder or condition. In some embodiments, a subject is susceptible to a disease, disorder, or condition. In some embodiments, a subject displays one or more symptoms or characteristics of a disease, disorder or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is an individual to whom diagnosis and/or therapy is and/or has been administered.

### Autoantibodies and Systemic Sclerosis

As used herein, the term “autoantibody” refers to an antibody produced by the immune system that is directed against one or more of a subject's own antigens, such as an epitope of a protein, a peptide, or a non-protein epitope. Such autoantibodies can be associated with autoimmune disease. Thus, in some embodiments, an autoantibody is obtained from a subject (or the therapeutic antibody used (e.g., affinity matured antibody) is derived from an autoantibody from a subject) with an autoimmune disease.

As used herein, the term “autoimmune disease” means a disease resulting from an immune response against a self-tissue or tissue component, including both self-antibody responses and cell-mediated responses. The term autoimmune disease, as used herein, encompasses organ-specific autoimmune diseases, in which an autoimmune response is directed against a single tissue, such as type I diabetes mellitus (TID), Crohn's disease, ulcerative colitis, myasthenia gravis, vitiligo, Graves' disease, Hashimoto's disease, Addison's disease and autoimmune gastritis and autoimmune hepatitis. The term autoimmune disease also encompasses non-organ specific autoimmune diseases, in which an autoimmune response is directed against a component present in several or many organs throughout the body. Such autoimmune diseases include, for example, rheumatoid diseases, systemic lupus erythematosus, progressive systemic sclerosis and variants, polymyositis and dermatomyositis. Additional autoimmune diseases include pernicious anemia including some of autoimmune gastritis, primary biliary cirrhosis, autoimmune thrombocytopenia, Sjögren's syndrome, multiple sclerosis and psoriasis.

Systemic sclerosis (SSc) is an autoimmune chronic fibrosing disease of unknown etiology that results in vasculopathy and multi-organ fibrosis. The disease is heterogeneous with a wide range of possible clinical manifestations that include skin thickening, interstitial lung disease (ILD), and Raynaud's phenomenon. The majority of SSc patients develop ILD, which has some clinical similarities with the progressive lung scarring seen in idiopathic pulmonary fibrosis (IPF). In some embodiments, telomere dysregulation can be observed in both SSc and IPF

Interstitial lung disease (ILD) can refer to pulmonary fibrosis, which shows scarring and inflammation of the tissue surrounding the lung's air sacs, blood vessels, and airways. In some embodiments, ILD can be related to an autoimmune disease (e.g, rheumatoid arthritis, scleroderma, polymyositis/dermatomyositis, lupus, and sarcoidosis).

Rheumatic diseases are disorders that affect joints, tendons, ligaments, bones, and muscles of a subject. In some embodiments, rheumatic disease can refer to musculoskeletal diseases. In some embodiments, examples of a rheumatic disease can include, but are not limited to, osteoarthritis, rheumatoid arthritis (RA), lupus, spondyloarthropathies (e.g, ankylosing spondylitis (AS) and psoriatic arthritis (PsA)), Sjögren's syndrome, gout, scleroderma, infectious arthritis, juvenile idiopathic arthritis, and polymyalgia rheumatica.

Provided herein are methods of identifying a disease in a subject that include: (a) providing a biological sample from a subject; and (b) detecting an autoantibody that targets an antigen of a telomerase/shelterin complex in the biological sample, thereby identifying a disease in the subject. In some embodiments, the disease is systemic sclerosis (SSc), idiopathic pulmonary fibrosis (IPF), or interstitial lung disease (ILD). In some embodiments, the disease is a rheumatic disease.

In some embodiments, the subject is a human. In some embodiments, the biological sample is blood or a blood fraction from the subject. In some embodiments, the biological sample is sera from the subject. In some embodiments, the autoantibody is a monoclonal antibody. In some embodiments, the autoantibody can target a telomere-associated protein. In some embodiments, the telomere-associated protein can include human telomerase reverse transcriptase (hTERT), telomerase TNA (TR or TERC), and dyskerin (DKC1). In some embodiments, the telomere-associated protein can include proteins of a shelterin complex, wherein the shelterin complex includes TRF1, TRF2, POT1, RAP1, TIN2, and TPP1.

### Telomerase/Shelterin Complex

As used herein, the term “telomerase” refers to a ribonucleoprotein that adds a species-dependent telomere repeat sequence to the 3′ end of telomeres. A telomere is a region of repetitive sequences at each end of the chromosomes of most eukaryotes. Telomeres protect the end of the chromosome from DNA damage or from fusion with neighboring chromosomes, wherein a telomerase is a reverse transcriptase enzyme that carries its own RNA molecule which is used as a template when it elongates telomeres. The molecular composition of the human telomerase complex includes two molecules each of human telomerase reverse transcriptase (TERT or hTERT in humans), telomerase TNA (TR or TERC), and dyskerin (DKC1), wherein the genes of telomere subunits can include TERT, TERC, DKC1, and TEP1.

As used herein, the term “shelterin complex” refers to a protein complex known to protect telomeres in many eukaryotes from DNA repair mechanisms, as well as to regulate telomerase activity. Shelterin complex includes six subunits: TRF1, TRF2, POT1, RAP1, TIN2, and TPP1, wherein the subunits bind along the length of the telomere. In some embodiments, the shelterin complex is crucial for both the maintenance of telomere structure and its signaling functions.

In some embodiments, an autoantibody can target an antigen of a telomerase/shelterin complex, wherein the antigen includes one or more of hTERT, TERF1, TERF2, POT1, TIN2L, TPP1, and RAP1. In some embodiments, an autoantibody can target TERF1.

### Biomarker for Systemic Sclerosis

In some embodiments, provided herein are methods of identifying a disease in a subject, wherein the method includes providing a biological sample from a subject, and detecting an autoantibody that targets an antigen of a telomerase/shelterin complex in the biological sample, thereby identifying a disease in the subject. In some embodiments, the disease can be systemic sclerosis, interstitial lung disease, and fibrotic lung disease. In some embodiments, the disease can be idiopathic pulmonary fibrosis (IPF). In some embodiments, the disease can be a rheumatic disease.

In some embodiments, the method includes detecting one or more autoantibodies. In some embodiments, the detecting includes detecting the presence or absence of an interaction between the autoantibody and the antigen. In some embodiments, an autoantibody can target one or more of the telomere-associated proteins. In some embodiments, an autoantibody can target a telomerase/shelterin complex, wherein the subject is identified to have a rheumatic disease. In some embodiments, the detecting comprises an immunoprecipitation assay, enzyme-linked immunosorbent assay (ELISA), or immunoblotting assay.

In some embodiments, the method further includes analyzing the biological sample to measure telomere length in a cell from the biological sample. In some embodiments, the telomere length is measured by performing qPCR, Flow-FISH, or both. In some embodiments, the cell is a leukocyte. In some embodiments, the cell is a peripheral blood mononuclear cell (PBMC). In some embodiments, the telomere length is short in leukocytes or lymphocytes, relative to the expected age-adjusted telomere length in a subject. In some embodiments, the telomere length is short in lymphocytes in a subject, wherein the subject has systemic sclerosis and TERF1 autoantibodies are detected from the subject. In some embodiments, a subject with TERF1 autoantibodies has shorter telomere lengths in lymphocytes, compared to a subject that does not have TERF1 autoantibodies.

In some embodiments, a subject is identified to have the disease when an autoantibody is detected in a biological sample from the subject. In some embodiments, the subject is identified to not have the disease when the autoantibody is not detected in the biological sample from the subject. In some embodiments, the subject is identified to have systemic sclerosis when a TERF1 autoantibody is detected in the biological sample from the subject.

### Therapeutic Applications

In some embodiments, provided herein are methods of treating a subject, wherein the subject is identified to have a disease when an autoantibody is detected in a biological sample from the subject, wherein the method includes administering a therapy for the disease. In some embodiments, provided herein are methods of treating a disease in a subject that include: (a) providing a biological sample from a subject; (b) detecting an autoantibody that targets an antigen present in a telomerase/shelterin complex in the biological sample, thereby identifying a disease in the subject; and (c) administering a treatment to the subject, wherein the treatment is predicted to be beneficial to the subject based on the presence or absence of the autoantibody. In some embodiments, the disease can be systemic sclerosis, interstitial lung disease, and fibrotic lung disease. In some embodiments, the disease can be idiopathic pulmonary fibrosis (IPF). In some embodiments, the disease can be a rheumatic disease.

In some embodiments, a treatment for systemic sclerosis can include anti-inflammatory medications (e.g., NSAIDs, corticosteroids), immunosuppressive therapy, drug therapy of vascular disease (e.g., calcium channel blockers, angiotensin converting enzyme (ACE) inhibitors, bosentan, and epoprostenol), or anti-fibrotic agents. In some embodiments, a treatment for interstitial lung disease or idiopathic pulmonary fibrosis can include, oxygen therapy, pulmonary rehabilitation, or drug therapy for pulmonary fibrosis (e.g., corticosteroid medications, pirfenidone, nintedanib, mycophenolate mofetil/mycophenolic acid, or azathioprine).

In some embodiments, the presence or absence of an autoantibody can determine the treatment to be administered to a subject. For example, an immunosuppressive therapy can be administered to a subject when the subject is identified to have an autoantibody. In some embodiments, a treatment can be administered to a subject having a disease as a combination therapy with one or more additional treatments.

### EXAMPLES

The disclosure is further described in the following examples, which do not limit the scope of the disclosure described in the claims.

### Example 1—Patient Cohorts

Sera were obtained from consecutive patients who met classification criteria for SSc at the Johns Hopkins (JH) and the University of California, San Francisco (UCSF) Scleroderma Centers. These two independent cohorts have similar databases and collect identical demographic and longitudinal clinical information including pulmonary function test data and organ-specific disease severity assessed by the Medsger Disease Severity Scale. Sera from healthy controls and patients with myositis, rheumatoid arthritis (RA), and IPF were also assayed.

Scleroderma patients were included in this study if they were over 18 years of age and met either the 2013 ACR/EULAR criteria for systemic sclerosis, the 1980 American College of Rheumatology (ACR) criteria, or had at least three of five features of the CREST syndrome (calcinosis, Raynaud's phenomenon, esophageal dysmotility, sclerodactyly, telangiectasias). Data included in both the JH and UCSF scleroderma databases included date of birth, sex, race, date of onset of first Raynaud's and first non-Raynaud's symptoms, history of cancer, and multiple measures of disease features and severity obtained at the first clinic visit and at 6 month intervals during follow-up visits. Organ-specific disease severity was defined by the Medsger Severity Scale. Skin thickness was quantified using the modified Rodnan skin thickness score (MRSS-[range 0-51]). Longitudinal pulmonary function test (PFT) data standardized by gender and age (diffusion capacity [DLCO], forced vital capacity [FVC]) and the right ventricular systolic pressure (RVSP) measured by echocardiogram were collected. Because clinical features of scleroderma change over time, the most extreme data points recorded (max/min, ever/never) were used to fully capture disease phenotype. PFT data obtained within one year of serum collection was also analyzed.

Medsger disease severity was defined as follows: Severe cardiac disease was defined by a Medsger score ≥1, severe lung disease was defined by a Medsger score ≥3, and severe muscle disease was defined by a Medsger score ≥1. The presence of myopathy was defined by the presence of muscle weakness (Medsger score ≥1 defined by power ≤4/5 in the upper or lower extremities) as well as the presence of at least one of the following: elevation in creatinine phosphokinase (CPK), abnormal electromyography (EMG), muscle edema visualized on magnetic resonance imaging (MRI), or myopathy confirmed on muscle biopsy.

A healthy control serum biobank was collected from donors in the Johns Hopkins rheumatology division. Participants were excluded from the healthy control cohort if they were pregnant, had a history of cancer, autoimmune disease, or chronic infection with hepatitis or HIV. The myositis cohort was randomly selected from all patients in the Johns Hopkins Myositis Center who met Bohan and Peter Criteria for a diagnosis of dermatomyositis or autoimmune necrotizing myopathy. Rheumatoid arthritis (RA) sera were obtained from a prospective cohort study of patients seen at the Johns Hopkins Arthritis center. The IPF cohort was obtained from the University of California, San Francisco.

To calculate the sample size of the healthy control cohort, it was planned to screen the entire scleroderma cohort of 442 patients. Using an estimated prevalence of TERF1 autoantibodies in scleroderma of 9% and assuming the prevalence of TERF1 autoantibodies among healthy controls is 1%, it was calculated that a sample size of at least 71 healthy controls would be needed to detect a difference using a power of 0.9 and an alpha of 0.05.

Discovery Cohort: Sera from a Subset of SSc Patients IP Telomerase and the Shelterin Proteins

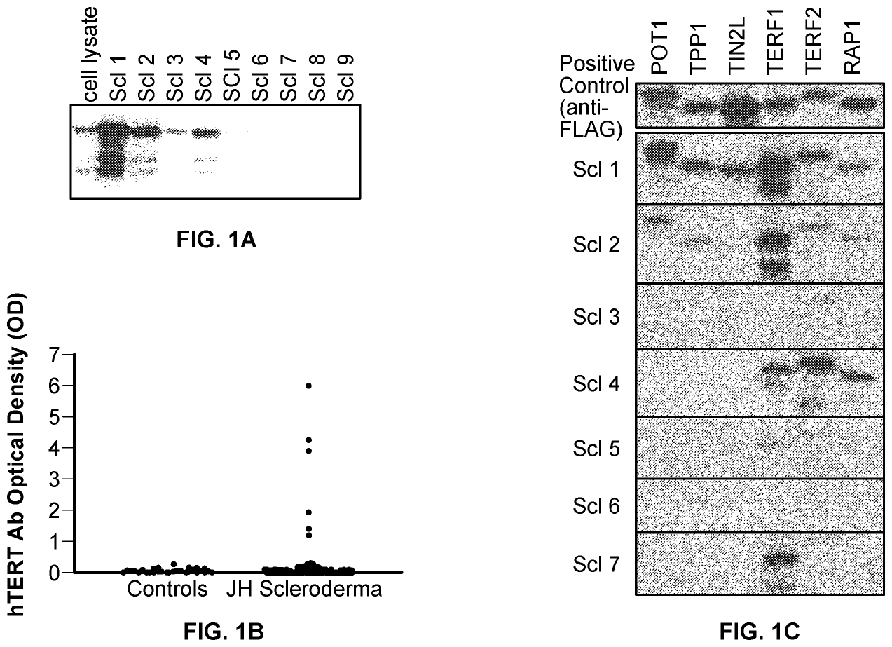

To test whether SSc patients have autoantibodies targeting telomerase (hTERT), 200 sera from the JH SSc Cohort were screened for these autoantibodies by IP using lysate made from HEK 293 cells overexpressing FLAG-tagged hTERT and telomerase RNA (hTR). The IPs were electrophoresed and hTERT was visualized by immunoblotting with anti-FLAG. Of the 200 JH SSc sera screened with this assay, 6 (3.0%) immunoprecipitated hTERT. hTERT autoantibodies were not identified in 30 healthy control sera (FIGS. 1A-1B).

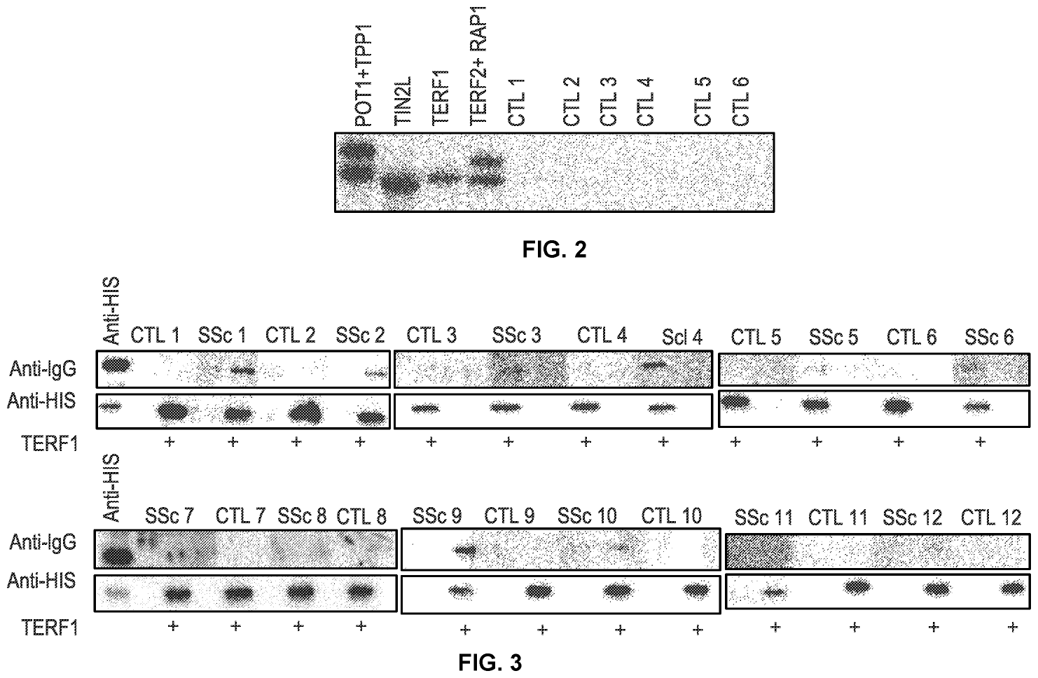

The same 200 sera from the JH SSc Cohort were screened for autoantibodies targeting the 6 shelterin proteins (POT1, TPP1, TIN2L, TERF1, TERF2, RAP1) by IP using 35S-methionine-labeled protein generated by IVTT as input. 7/200 (3.5%) SSc sera immunoprecipitated either hTERT or one of the shelterin proteins, and 6 of these patients had multiple telomere-associated autoantibodies (FIG. 1C). In contrast, 0/30 healthy controls had a shelterin autoantibody (FIG. 2). None of the 7 patients with telomere-associated autoantibodies had autoantibodies targeting DKC1 or NHP2.

### Validation Cohort: TERF1 Autoantibodies Detected by ELISA in the JH and UCSF SSe Cohorts

As TERF1 was the most common of the shelterin autoantibodies and overlapped with multiple other telomere-associated autoantibodies, an ELISA was developed to screen for TERF1 autoantibodies. 5/6 patients with TERF1 autoantibodies identified by IVTT IP were positive by ELISA (FIGS. 1A-1C). In total, the ELISA detected TERF1 autoantibodies in 22/200 (11.0%) of the JH Cohort. As a validation cohort, 242 sera from the UCSF SSc Cohort was screened by ELISA and identified TERF1 autoantibodies in 18/242 (7.4%) patients. Table 1 includes demographic and clinical features of both cohorts. Of the 40 patients total with TERF1 autoantibodies identified by ELISA, 7/40 (18%) were positive by TERF1 IVTT IP. While the ELISA likely detected more positive sera compared to IVTT IP because of differences in TERF1 protein conformation used in the assays, a third assay was set up (immunoblotting of recombinant TERF1 protein) to better address the issue of the discrepant TERF1 autoantibody readouts. Using this, the presence of TERF1 autoantibodies in 25/32 (78%) sera that were ELISA-positive but IP-negative was confirmed using patient sera to immunoblot TERF1 protein (FIG. 3). The number of healthy controls screened was expanded to 78 and it was found that the prevalence of TERF1 autoantibodies among SSc patients in both cohorts (40/442 [9.0%]) was significantly higher compared to healthy controls (1/78 [1.3%]), p=0.01).

TABLE 1

| | JH SSc Cohort | | UCSF SSc Cohort | | |
|---|---|---|---|---|---|
| | (n = 200) | | (n = 242) | | P value |
| Age (years), mean (SD) | 57.9 | (13.4) | 54.6 | (13.2) | p = 0.009 ** |
| Sex | | | | | |
| Female, n [%] | 171 | [85%] | 207 | [86%] | P = 1.0 |
| Male, n [%] | 29 | [15%] | 35 | [14%] | |
| Race | | | | | |
| Caucasian, n [%] | 153/198 | [77%] | 142/241 | [59%] | P < 0.0001 *** |
| African American, n [%] | 36/198 | [18%] | 23/241 | [10%] | |
| Asian or Indian, n [%] | 9/198 | [5%] | 76/241 | [32%] | |
| SSc Type | | | | | |
| Limited, n [%] | 127 | [64%] | 160 | [66%] | P = 0.62 |
| Diffuse, n [%] | 73 | [36%] | 82 | [34%] | |
| Disease Duration at time of bleed | | | | | |
| From onset of RP, median | 12.5 | (6.6-21.4) | 11.1 | (5.4-20.0) | P = 0.09 |
| (IQR) | | | | | |
| From onset of non-RP | 12.1 | (6.1-18.2) | 9.4 | (4.3-16.3) | P = 0.001 ** |
| symptom, median (IQR) | | | | | |
| Autoantibody status | | | | | |
| Centromere, n [%] | 62/199 | [31%] | 60/241 | [25%] | P = 0.16 |
| U1RNP, n [%] | 17/200 | [9%] | 19/237 | [8%] | P = 0.86 |
| Scl70, n [%] | 44/199 | [22%] | 64/241 | [27%] | P = 0.32 |
| RNA polymerase III, n [%] | 39/199 | [20%] | 43/237 | [18%] | P = 0.81 |
| Ku, n [%] | 11/199 | [6%] | | | |
| No SSc-specific Ab, n [%] | 64/199 | [32%] | 82/241 | [34%] | P = 0.69 |
| Clinical Features | | | | | |
| History of cancer (ever), n [%] | 38/200 | [19%] | 39/242 | [16%] | P = 0.45 |
| Mortality, n [%] | 16/242 | [7%] | 6/200 | [3%] | P = 0.12 |
| Inflammatory arthritis (ever), n [%] | 40/200 | [20%] | 55/242 | [23%] | P = 0.56 |
| Digital ulceration or gangrene | 47/200 | [24%] | 100/242 | [41%] | P < 0.0001 *** |
| (ever), n [%] | | | | | |
| SSc renal crisis (ever), n [%] | 5/200 | [3%] | 11/242 | [5%] | P = 0.31 |
| Myopathy (ever), n [%] | 43/200 | [22%] | 23/242 | [10%] | P = 0.0005 *** |
| Max MRSS, mean (SD) | 10.9 | (10.4) | 6.6 | (7.2) | P < 0.0001 *** |
| Severe muscle disease (ever), | 51/200 | [26%] | 18/242 | [7%] | P < 0.0001 *** |
| n [%] | | | | | |
| Severe heart disease (ever), n | 53/194 | [27%] | 52/242 | [22%] | P = 0.18 |
| [%] | | | | | |
| Severe lung disease (ever), n | 59/196 | [30%] | 92/242 | [38%] | P = 0.09 |
| [%] | | | | | |
| Max RVSP (mmHg), mean | 36.2 | (11.8) | 38.4 | (21.3) | P = 0.17 |
| (SD) | | | | | |
| Min DLCO % predicted, mean | 64.8 | (20.5) | 52.3 | (21.3) | P < 0.0001 *** |
| (SD) | | | | | |
| Min FVC % predicted, mean | 74.4 | (19.2) | 70.7 | (22.6) | p = 0.07 |
| (SD) | | | | | |

### Example 2—Immunoprecipitation Assays for Autoantibody Detection

### Cell Lysate Immunoprecipitation

To determine if patient sera contain autoantibodies targeting hTERT, an immunoprecipitation (IP) assay was developed using a cell lysate overexpressing telomerase. A cell line overexpressing the telomerase RNA component (hTR) and FLAG-tagged human telomerase (hTERT) was generated using a Flp-In T-Rex 293 cell line per the manufacturer's instructions (Thermo Fisher). 50 ug of cell lysate was pre-cleared with protein A beads in NP40 Lysis Buffer (NP40 Lysis buffer used: 1% NP40, 20 mM Tris [pH 7.4], 150 mM NaCl, 1 mM EDTA supplemented with protease inhibitor cocktail) and immunoprecipitated with 1 ul patient serum. Immunoprecipitates were electrophoresed on SDS-PAGE gels blotted with anti-FLAG antibody (Millipore, Sigma) and visualized using enhanced chemiluminescence (ThermoFisher) in a FluorChem M chemi-luminescence imager (ProteinSimple). The data were quantitated by densitometric scanning of the blots and analyzed using ImageJ. Each sample set was calibrated with the same positive reference IP that was run on each blot. The cut-off for a positive autoantibody was defined as the mean+4 SD of the healthy controls.

### IP Using 35S-Methionine-Labeled Proteins

Complementary DNAs for human POT1, TPP1, TIN2L, TERF1, TERF2, RAP1, NHP2 and DKC1 (GenScript) were used to generate 35S-methioinine-labeled proteins by in vitro transcription and translation (IVTT) per the manufacturer's protocol (Promega). The radiolabeled proteins were immunoprecipitated with patient sera in Lysis Buffer, and the products were electrophoresed on SDS-PAGE gels and visualized by fluorography.

### Example 3—TERF1 ELISA

ELISA plates were coated overnight at 4° C. with 200 ng/well of recombinant full-length TERF1 protein (Sino Biological). Patient sera were used at 1:200 dilution and secondary antibodies were horseradish peroxidase-labeled. The color was developed using SureBlue peroxidase reagent (Seracare Life Sciences) and the absorbance was read at 450 nm. The same positive reference serum (with an optical density (OD) in the linear range) was included on every plate as a calibrator. The cutoff for autoantibody positivity was set as the mean plus 4 standard deviations of 50 healthy controls. All positive sera were re-tested by ELISA alongside an uncoated well; ODs of the uncoated wells were subtracted from those obtained with TERF1 coated wells.

### TERF1 Autoantibodies in Other Rheumatic Diseases

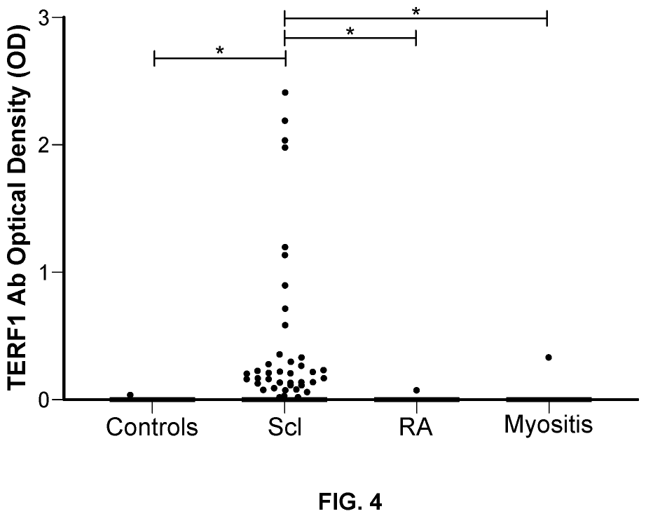

To determine the specificity of TERF1 autoantibodies for systemic sclerosis, 60 RA and 60 myositis sera were screened for TERF1 autoantibodies by ELISA. The mean age of the systemic sclerosis, RA, and myositis cohorts were similar (Table 2 and 3). In each of the RA and myositis cohorts, 1/60 (1.7%) patients had a positive TERF1 autoantibody, which was similar to healthy controls (FIG. 4). TERF1 autoantibodies were significantly more frequent among SSc patients (JH and UCSF combined) compared to RA or myositis (40/442 [9.0%] vs 1/60 [1.7%], p=0.05 in each case).

TABLE 2

| | Demographics and myositis clinical | |
|---|---|---|
| | features (n = 60) | |
| Age (years), mean (SD) | 52.6 | (18.0) |
| Sex, female, n [%] | 49 | [82%] |
| Disease duration (years) from onset | 1.8 | (1.1-4.6) |
| of symptoms to blood draw, | | |
| median (IQR) | | |
| Type | | |
| Dermatomyositis, n [%] | 46 | [77%] |
| Necrotizing myositis, n [%] | 14 | [23%] |
| Max creatine kinase (CK), median | 879 | (116-3498) |
| (IQR) | | |
| Autoantibodies | | |
| TIF-1 gamma | 8/52 | [15%] |
| NXP-2 | 6/52 | [12%] |
| U1RNP | 1/52 | [2%] |
| MDA5 | 2/52 | [4%] |
| Mi2 | 3/52 | [6%] |
| Jo-1 | 6/52 | [12%] |
| PL-12 | 1/52 | [2%] |
| Ro-52 | 25/52 | [48%] |
| PM-75 | 6/52 | [12%] |
| PM-100 | 6/52 | [12%] |
| PM-Scl | 3/45 | [7%] |
| SRP | 11/48 | [23%] |
| HMGCR | 5/48 | [10%] |

TABLE 3

| | Demographics and | |
|---|---|---|
| | rheumatoid arthritis | |
| | clinical features (n = 60) | |
| Age (years), mean (SD) | 55.6 | (12.6) |
| Sex, female, n [%] | 47/60 | [78%] |
| Race, white, n [%] | 51/60 | [85%] |
| Disease duration (years) from onset | 8 | (4-13) |
| of symptoms to blood draw, | | |
| median (IQR) | | |
| Autoantibodies | 34/57 | [60%] |
| Rheumatoid factor positive, n [%] | 36/59 | [61%] |
| Cyclic citrullinated peptide | | |
| antibody positive, n [%] | | |
| Erosive disease | 27 | [46%] |
| CDAI score, median (IQR) | 6.9 | (2.5-14) |

### Example 4—Immunoblotting Assays and Other Autoantibody Assays

TERF1 recombinant protein (His-tagged, 200 ng/lane; Sino Biological) was electrophoresed on 10% SDS-PAGE gels, followed by transfer to nitrocellulose membranes. All washes were performed with TBS-0.05% Tween 20 (TBST). Blocking was performed for 1 hour at room temperature with 5% milk in TBST, followed by incubation with patient sera (diluted 1:2000 in 5% milk-TBST overnight at 4° C.). After incubation with horseradish peroxidase-labeled secondary antibody (anti-human IgG), TERF1 antibodies were visualized with enhanced chemiluminescence (Thermo Scientific). For each serum, immunoblots were reprobed with an anti-His monoclonal antibody (GenScript) as a loading control. Images were generated using a ProteinSimple FluorChem M digital imager

The JH SSc sera were screened for autoantibodies targeting SSc-associated autoantibodies using the line immunoblot platform (EuroImmun: Systemic Sclerosis [Nucleoli] profile). UIRNP autoantibodies were assayed using a commercially available ELISA (Inova Diagnostics, CA). Euroimmun results were considered positive per the manufacturer's guidelines. Autoantibodies in the UCSF cohort were derived from clinically indicated commercial testing.

For the Euroimmune autoantibody profile, indeterminates were considered negative. A positive anti-centromere antibody was defined as reactivity to either centromere A (CENP-A) or centromere B (CENP-B). A positive RNA polymerase III antibody was defined as reactivity to either the RP11 or RP155 subunits.

### Example 5—TERF1 Autoantibodies and Telomere Length in Leukocytes

Two assays were used to measure telomere length: (i) A PCR-based assay measured telomere length in peripheral leukocytes from the UCSF SSc Cohort; and (ii) Flow-FISH was used to measure telomere length in banked peripheral blood mononuclear cells (PBMC's) prospectively collected from a subset of the JH SSc Cohort with and without TERF1 autoantibodies. Flow-FISH was done on all samples in batch. Telomere lengths were compared with a validated nomogram of telomere length among healthy controls.

A PCR-based assay was used to measure telomere length: Peripheral blood genomic DNA was extracted using Centra Puregene cell kit (Qiagen, Valencia, CA, USA). DNA quality was assessed by agarose gel electrophoresis and degraded DNA was discarded. Peripheral blood leukocyte telomere length was measured in triplicate using quantitative uniplex qPCR relative to the housekeeping gene acidic ribosomal phosphoprotein 36B4.

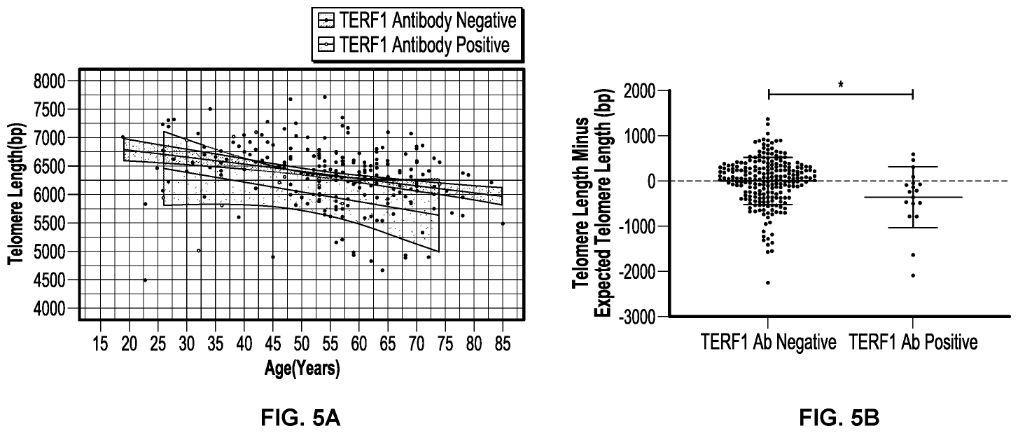

It was further determined if autoantibodies targeting the telomerase/shelterin complex are associated with abnormalities in telomere length. Telomere length was measured by qPCR in peripheral leukocytes from all UCSF SSc patients using the same banked blood draw from which the TERF1 autoantibodies were assayed. FIG. 5A shows telomere length plotted by age for all patients with and without TERF1 autoantibodies. Given that telomeres shorten with a constant linear rate in middle-age, each patient's expected telomere length was calculated using a linear regression model based on the relationship between age and telomere length among the TERF1 autoantibody-negative patients (expected telomere length (bp)=7028-12.62 [years of age]). The difference between the patient's telomere length and the expected telomere length was then calculated for each patient. Compared to patients without TERF1 autoantibodies, significantly more patients with TERF1 autoantibodies had a shorter telomere length than the expected age-adjusted telomere length (14/18 [78%] vs 96/224 [43%]), p=0.006). Furthermore, the difference between the patient telomere length and the expected age-adjusted telomere length was significantly more negative for patients with TERF1 autoantibodies compared to patients without TERF1 autoantibodies (median −230 [IQR −572 to −18] vs 53 [−272 to 304] bp, p=0.01, Wilcoxon rank-sum) (FIG. 5B).

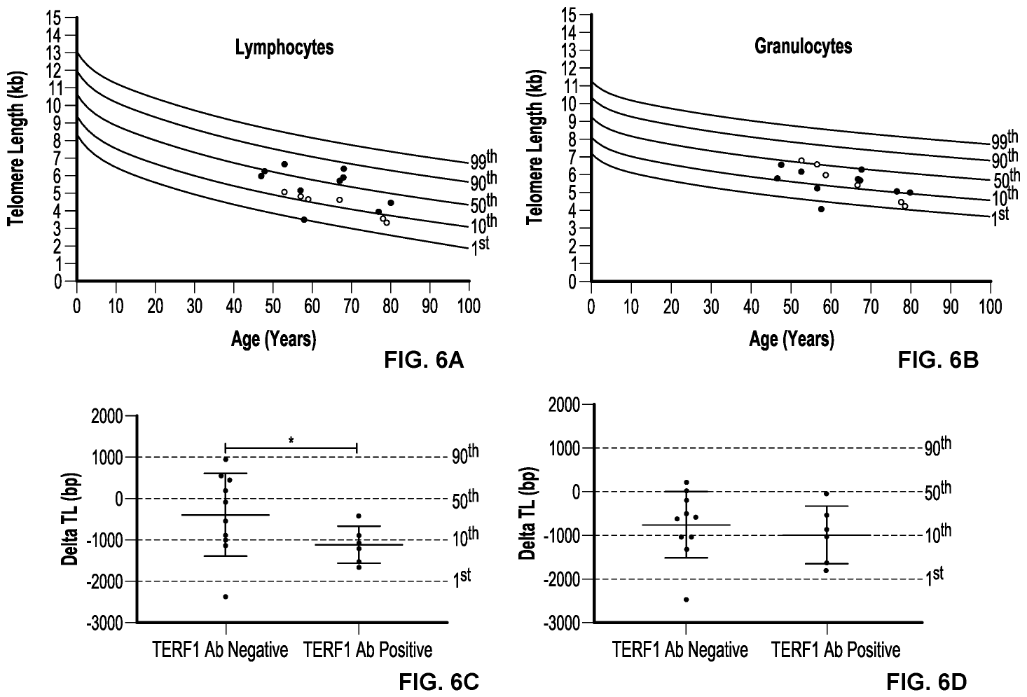

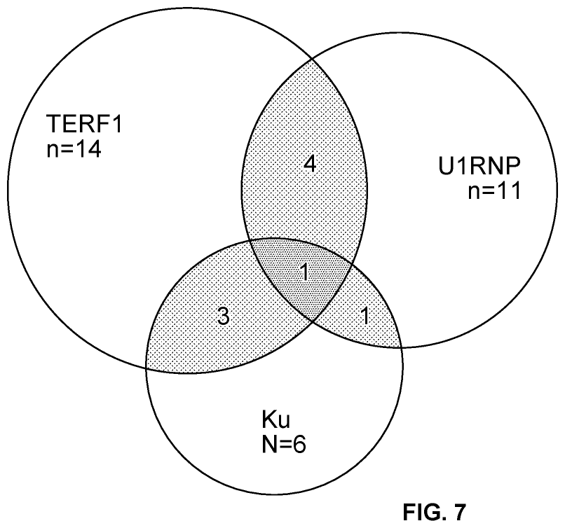

The association between TERF1 autoantibodies and short telomeres was then confirmed in the JH SSc Cohort using the Flow-FISH assay, which is known to be more accurate, reproducible, sensitive and specific compared to qPCR and can simultaneously differentiate telomere length in lymphocytes and granulocytes. 6 patients with TERF1 autoantibodies and 10 patients without TERF1 autoantibodies were identified who presented for routine clinical visits and agreed to donate PBMC's. ELISAs performed on serum collected concurrently were used to determine TERF1 autoantibody status. Telomere length was measured on PBMC's using Flow-FISH. The delta TL (telomere length), which is the difference between the patient's telomere length and the median telomere length for a healthy person of the same age, was significantly more negative for the TERF1 autoantibody-positive patients compared to the TERF1 autoantibody-negative patients in lymphocytes (median −1132 [IQR −1552 to −996] vs −254 [−950 to 464] bp, p=0.03, Wilcoxon rank-sum). This difference was not observed in granulocytes (median −706 [IQR −1686 to 22] vs −829 [−1122 to −446] bp, p=0.8, Wilcoxon rank-sum) (FIGS. 6A-6D). The two patients with the highest titer hTERT autoantibodies both had telomere lengths below the 10th percentile in lymphocytes and granulocytes.

### Example 6—Statistics

Fisher's exact test was used to evaluate differences in the frequency of TERF1 autoantibodies between different patient cohorts. The various demographic, clinical, and serologic features of systemic sclerosis, as well as differences in telomere length, were compared between the TERF1 autoantibody-negative and -positive patients using the Wilcoxon rank-sum test or student's t-test for continuous variables and the Fisher's exact test for dichotomous variables. All statistical analyses were 2-sided and were conducted using JMP Version 9 (SAS Institute Inc). p<0.05 was considered significant.

### Example 7—Clinical and Serologic Associations with TERF1 Autoantibodies in Systemic Sclerosis

After identifying the existence of TERF1 autoantibodies in SSc and demonstrating an association of these autoantibodies with short telomeres in lymphocytes, the associated clinical and serological features were explored (Table 4). The JH and UCSF SSc Cohorts use standardized clinical definitions with harmonization in clinical data acquisition, enabling the evaluation of clinical associations for all 40 TERF1 autoantibody-positive and 402 TERF1 autoantibody-negative patients. The length of clinical follow-up was similar between patients with and without TERF1 autoantibodies (6.8±6.2 vs 6.0±6.0 years, p=0.46). Patients with TERF1 autoantibodies tended to be slightly younger (52.6±13.7 vs 56.4±13.3 years, p=0.10). The presence of TERF1 autoantibodies was significantly associated with a history of severe lung disease (OR 2.4 [CI 1.2-4.8], p=0.04) and a lower percent predicted diffusion capacity (DLCO) within one year of serum collection (58.0 vs 67.9, p=0.02, student's t-test). There was also an association with a history of severe muscle disease (OR 3.0 [CI 1.4-6.1], p=0.005) and inflammatory arthritis (OR 2.1 [CI 1.1-4.3], p=0.04). Non-white race was strongly associated with severe lung disease (OR 2.3 [CI 1.5-3.5], p<0.0001) and was also associated with the presence of TERF1 autoantibodies (OR 2.5 [CI 1.3-4.8], p=0.005). The association between TERF1 autoantibodies and severe lung disease was not statistically significant after adjusting for race (OR 1.73 [CI 0.88-3.4] p=0.11).

TERF1 autoantibodies were associated with UIRNP autoantibodies in the combined cohorts (OR 4.8 [CI 2.1-10.8], p=0.0006) and Ku autoantibodies in the JH cohort (OR 5.4 [CI 1.4-20.2], p=0.02) (Table 4, FIG. 7). Ku autoantibody status was not available for the UCSF cohort. There was no association with the frequent SSc-specific autoantibodies anti-Scl-70, anti-centromere, or anti-RNA polymerase III. Absence of these SSc-specific autoantibodies was observed slightly more frequently in patients with TERF1 autoantibodies compared to patients without TERF1 autoantibodies (17/40 [42%] vs 129/400 [32%], p=0.22), although this difference was not significant.

TABLE 4

| | TERF1 Ab positive | | TERF1 Ab negative | | |
|---|---|---|---|---|---|
| | (n = 40) | | (n = 402) | | p-value |
| Age (years), mean (SD) | 52.6 | (13.7) | 56.4 | (13.3) | P = 0.10 |
| Sex, female, n [%] | 34 | [85%] | 344 | [86%] | P = 1.0 |
| Race, Caucasian, n [%] | 19/40 | [48%] | 276/399 | [69%] | P = 0.008 ** |
| African American, n [%] | 10/40 | [25%] | 49/399 | [12%] | |
| Asian, n [%] | 11/40 | [28%] | 74/399 | [19%] | |
| SSc Type, limited, n [%] | 24 | [60%] | 263 | [65%] | P = 0.49 |
| Disease duration | | | | | |
| From onset of RP, median | 13.9 | (7.2-22.5) | 11.6 | (5.9-20.6) | P = 0.08 |
| (IQR) | | | | | |
| From onset of non-RP | 12.2 | (8.4-17.5) | 10.4 | (4.5-17.1) | P = 0.08 |
| symptom, median (IQR) | | | | | |
| Autoantibody status | | | | | |
| Centromere, n [%] | 10/40 | [25%] | 112/400 | [28%] | P = 0.85 |
| U1RNP, n [%] | 10/40 | [25%] | 26/397 | [7%] | P = 0.0006 *** |
| Scl70, n [%] | 11/40 | [28%] | 97/400 | [24%] | P = 0.70 |
| RNA polymerase III, n [%] | 5/39 | [13%] | 77/397 | [19%] | P = 0.39 |
| Ku, n [%] | 4/22 | [18%] | 7/177 | [4%] | P = 0.02 * |
| No SSc-specific Ab, n [%] | 17/40 | [42%] | 129/400 | [32%] | P = 0.22 |
| Clinical features (ever, max/min) | | | | | |
| History of cancer (ever), n [%] | 7/40 | [18%] | 70/402 | [17%] | P = 1.0 |
| Mortality, n [%] | 1/40 | [3%] | 21/402 | [5%] | P = 0.71 |
| Inflammatory arthritis (ever), n | 14/40 | [35%] | 81/402 | [20%] | P = 0.04 * |
| [%] | | | | | |
| Digital ulceration or gangrene | 12/40 | [30%] | 140/402 | [35%] | p = 0.60 |
| (ever), n [%] | | | | | |
| SSc renal crisis (ever), n [%] | 1/40 | [3%] | 15/402 | [4%] | P = 1.0 |
| Myopathy (ever), n [%] | 8/40 | [20%] | 58/402 | [14%] | P = 0.35 |
| Max MRSS, mean (SD) | 8.0 | (9.3) | 8.6 | (9.0) | P = 0.44 |
| Severe muscle disease (ever), | 13/40 | [33%] | 56/402 | [14%] | P = 0.005 ** |
| n [%] | | | | | |
| Severe heart disease (ever), n | 14/39 | [36%] | 91/397 | [23%] | P = 0.08 |
| [%] | | | | | |
| Severe lung disease (ever), n | 20/40 | [50%] | 131/398 | [33%] | P = 0.04 * |
| [%] | | | | | |
| Max RVSP (mmHg), mean | 39.9 | (20.1) | 37.3 | (17.7) | P = 0.46 |
| (SD) | | | | | |
| Min DLCO % predicted, mean | 53.0 | (20.6) | 58.4 | (21.9) | P = 0.13 |
| (SD) | | | | | |
| Min FVC % predicted, mean | 66.5 | (20.4) | 72.9 | (21.2) | P = 0.07 |
| (SD) | | | | | |
| PFT's within one year of bleed date | TERF1 Ab positive | | TERF1 Ab negative | | |
| | (n = 34) | | (n = 354) | | |
| DLCO % predicted, mean (SD) | 58.0 | (22.5) | 67.9 | (23.4) | P = 0.02 * |
| FVC % predicted, mean (SD) | 75.0 | (21.3) | 80.2 | (20.5) | P = 0.18 |

### Example 8—TERF1 Autoantibodies in Idiopathic Pulmonary Fibrosis

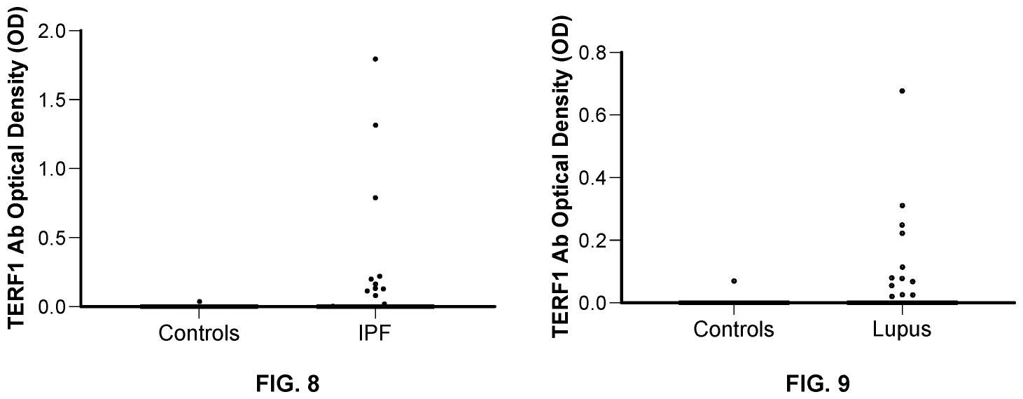

To address whether TERF1 autoantibodies might be present in other syndromes in which telomere dysfunction and lung fibrosis are prominent, 152 patients with IPF were screened and TERF1 autoantibodies were identified in 11/152 (7.2%) patients, compared to only 1/78 (1.3%) positives among healthy controls (p=0.06) (FIG. 8). Further details on the IPF cohort are in Table 5. The patient in the IPF cohort with the highest TERF1 autoantibody titer had a positive ANA (1:160, speckled) and subsequently developed symptoms of SSc approximately 2 years later. It was determined that this patient most likely had systemic sclerosis-ILD rather than IPF, although the TERF1 autoantibody had preceded the other clinical features of systemic sclerosis. The other IPF patients with TERF1 autoantibodies did not have a positive ANA and have not developed any features of a systemic autoimmune disease.

TABLE 5

| | TERF1 Ab positive | | TERF1 Ab negative | | |
|---|---|---|---|---|---|
| | (n = 11) | | (n = 141) | | p-value |
| Age (years), mean (SD) | 71.8 | (5.6) | 70.9 | (8.1) | P = 0.65 |
| Sex, female, n [%] | 1 | [9%] | 27 | [19%] | P = 0.69 |
| Race, Caucasian, n [%] | 9 | [82%] | 124 | [88%] | P = 0.63 |
| Disease duration (years) since | 4.6 | (2.2-7.3) | 3.4 | (1.4-5.3) | P = 0.20 |
| blood draw, median (IQR) | | | | | |
| Deceased | 8 | [73%] | 101 | [72%] | P = 1.0 |
| History of smoking | 7 | [63%] | 103 | [73%] | P = 0.50 |
| Presence of usual interstitial | 10 | [91%] | 119 | [86%] | P = 1.0 |
| pneumonia (UIP) | | | | | |
| PFT's within one year of bleed date | | | | | |
| DLCO % predicted, mean (SD) | 53.5 | (9.4) | 48.1 | (17.2) | p = 0.11 |
| FVC % predicted, mean (SD) | 67.6 | (13.9) | 72.0 | (17.5) | P = 0.37 |
| FEV1 % predicted, mean (SD) | 76.4 | (15.1) | 81.3 | (19.7) | P = 0.35 |

What is claimed is:

1. A method of identifying a disease in a subject, the method comprising:
(a) providing a biological sample from a subject; and
(b) detecting an autoantibody that targets an antigen present in a telomerase/shelterin complex in the biological sample, thereby identifying a disease in the subject.

2. A method of treating a disease in a subject, the method comprising:
(a) providing a biological sample from a subject;
(b) detecting an autoantibody that targets an antigen present in a telomerase/shelterin complex in the biological sample, thereby identifying the disease in the subject; and
(c) administering a treatment to the subject, wherein the treatment is predicted to be beneficial to the subject based when the presence of the autoantibody is detected.

3. The method of claim 1 or 2, wherein the disease is systemic sclerosis (SSc), idiopathic pulmonary fibrosis (IPF), or interstitial lung disease (ILD).

4. The method of any one of claims 1-3, wherein the disease is a rheumatic disease.

5. The method of any one of claims 2-4, wherein the treatment comprises administration of a therapy selected from an anti-inflammatory medication, an immunosuppressive therapy, a drug therapy of vascular disease, an anti-fibrotic agent, an oxygen therapy, pulmonary rehabilitation, or a drug therapy for pulmonary fibrosis, and combinations thereof.

6. The method of any one of claims 1-5, wherein the subject is a human.

7. The method of any one of claims 1-6, wherein the biological sample is blood or a blood fraction obtained from the subject.

8. The method of any one of claims 1-7, wherein the biological sample is sera obtained from the subject.

9. The method of any one of claims 1-8, wherein the autoantibody is a monoclonal antibody.

10. The method of any one of claims 1-9, wherein the antigen comprises one or more of hTERT, TERF1, TERF2, POT1, TIN2L, and RAP1.

11. The method of any one of claims 1-10, wherein the method comprises detecting two or more autoantibodies.

12. The method of any one of claims 1-11, wherein the detecting step (b) comprises detecting the presence or absence of an interaction between the autoantibody and the antigen.

13. The method of any one of claims 1-12, wherein the detecting step (b) comprises an immunoprecipitation assay, an enzyme-linked immunosorbent assay (ELISA), or an immunoblotting assay.

14. The method of any one of claims 1-13, further comprising analyzing a second biological sample from the subject to measure telomere length in a cell from the second biological sample.

15. The method of claim 14, wherein the biological sample and the second biological sample are the same.

16. The method of any one of claims 14-15, wherein the telomere length is measured by performing qPCR, Flow-FISH, or both.

17. The method of any one of claims 14-16, wherein the cell is a leucocyte.

18. The method of any one of claims 14-16, wherein the cell is a peripheral blood mononuclear cell (PBMC).

19. The method of any one of claims 1-18, wherein the subject is identified to have the disease when the autoantibody is detected in the biological sample from the subject.

20. The method of any one of claims 1-18, wherein the subject is identified to not have the disease when the autoantibody is not detected in the biological sample from the subject.
